(19) United States
(12) Patent Application Publication
(10) Pub. No.: US 2019/0000989 A1
(43) Pub. Date: Jan. 3, 2019

(54) BISPECIFIC TARGETING AGENTS AND METHODS FOR THEIR PREPARATION

(71) Applicant: STC Biologics Inc., Newton, MA (US)

(72) Inventors: Magdalena Leszczyniecka, Newton, MA (US); Thomas J. Schneider, Newton, MA (US); Lisa Ganley-Leal, Newton, MA (US)

(21) Appl. No.: 16/069,119

(22) PCT Filed: Jan. 11, 2017
(86) PCT No.: PCT/US17/13006

§ 371 Date: Jul. 10, 2018

Related U.S. Application Data
(60) Provisional application No. 62/277,249, filed on Jan. 11, 2016.

(51) Int. Cl. A61K 47/69; A61K 47/68; A61K 38/20; C07K 16/46

(52) U.S. Cl. CPC A61K 47/6913; A61K 47/6879; A61K 47/6873; A61K 47/6849; A61K 47/6855; C07K 2317/31; A61K 47/6811; A61K 47/6813; A61K 38/2013; A61K 38/2086; C07K 16/468; A61K 47/6865

(57) ABSTRACT

The present invention is directed to a platform for new bispecific targeting agents using liposomes or nanoparticles as linkers. The bispecific targeting agent can bind two different cell types, each via a separate targeting moiety. Said targeting agents can be used to induce specific biological effects in the cells such as cell proliferation or cell activation which can be used in some instances to destroy the other bound cells. Any cell that can be targeted can be subject to targeting. For example, the cell types that may be recruited by the bispecific targeting agent may be both human or one of the cells may be human and the other an infected cell or it can be an infectious agent. The platform is based on an empty nanoparticle or liposome conjugated to two or more targeting moieties, bound to the nanoparticle/liposome at defined ratios that may be other than 1:1. Such, compositions provide for specific binding to each cell type. This bispecific targeting agent can further be linked to growth factors or cytokines to further potentiate the effect of the bispecific targeting agent as a therapeutic or to exert a specific biologic effect on one or both cells being targeted.

### FIELD OF INVENTION

The present invention is directed to a platform for new bi- and multi-specific targeting agents using liposomes or nanoparticles as linkers. The targeting agents can bind at least two different cell types, each via a separate targeting moiety. Said targeting agents can be used to induce specific biological effects in the cells such as cell proliferation or cell activation which can be used in some instances to destroy the other bound cells.

### BACKGROUND OF THE INVENTION

Uniting two antigen binding sites of different specificity into a single construct, bispecific antibodies have the ability to bring together two discreet antigens with exquisite specificity. This gives them great potential for use as therapeutic agents. This potential was recognized early on, leading to a number of approaches for obtaining such bispecific antibodies using various biologic linkers Bispecific antibodies have been made by fusing two hybridomas, each capable of producing a different immunoglobulin. When successful the resulting hybrid-hybridoma, or quadroma, produces antibodies bearing the antigen specificity of both parent hybridomas (Milstein et al. (1983), Nature 305:537).

Largely due to technical difficulties frequently encountered with this early approach, later work focused on creating antibody constructs by joining two scFv antibody fragments while omitting the Fc portion present in full immunoglobulins. Each scFv unit in such constructs consisted of one variable domain from each of the heavy (VH) and light (VL) antibody chains, joined together via a synthetic polypeptide linker, the latter often being genetically engineered so as to be minimally immunogenic while remaining maximally resistant to proteolysis. Respective scFv units were joined with different biologic linkers, but not liposomes, including incorporation of a short (usually less than 10 amino acids) polypeptide spacer bridging the two scFv units, thereby creating a bispecific single chain antibody. Bispecific single chain antibodies have nucleotide sequences encoding the four V-domains, two linkers and one spacer can be incorporated into a suitable host expression organism under the control of a single promoter. However, problems can be encountered with expression because the multidomain recombinant proteins may not be expressed or fold correctly.

Nevertheless, when successful, remarkable results have been obtained using such bispecific antibodies designed for the treatment of malignancies (Mack, J. Immunol. (1997), 158:3965-70; Mack, PNAS (1995), 92:7021-5; Kufer, Cancer Immunol. Immunother. (1997), 45:193-7; Löffler, Blood (2000), 95:2098-103) and non-malignant diseases (Brühl, J. Immunol. (2001), 166:2420-6). However, bispecific single chain antibodies must fulfill additional requirements. In order to achieve the desired activity, the bispecific antibody should properly and stably fold, something that often proves unrealizable because of their complex multi domain structure. Often less conventional, more cumbersome and costly eukaryotic—even mammalian—expression systems are required. These systems complicate the production of bispecific single chain antibodies and can reduce product yield to the levels that are lower than desired for therapeutic application.

In the event that a bispecific antibody is intended for therapeutic use, it is desirable to produce high amounts of fully functional targeting arms (antibodies, antibody fragments, single chains) and in the desired functional form. The production of functionally active antibody becomes especially critical when producing bispecific agents of which one portion is able to activate and recruit the cytotoxic potential of human immune effector cells; because, an antibody devoid of functional activity can not lead to the desired activation of human immune effector cells, while a bispecific antibody which is functionally active, albeit not in the desired manner, as may be the case when the bispecific antibody is produced in a heterogeneous form containing multiple isomers, may activate and recruit the cytotoxic potential of human immune effector cells in unforeseeable and/or unintended manners.

New bi- and multi-specific targeting agents are needed that have fully functional targeting moieties as are convenient methods for their preparation. Ideally the targeting moieties can be produced using well defined production methods using either eukaryotic (e.g., mammalian) or bacterial cells. Ideally the ratio of targeting moieties can be adjusted to a wide range of values. This would overcome a limitation present with current bispecific targeting agents where the targeting moieties are present in a 1:1 ratio. By changing the ratio of one targeting arm to the second the affinity of the bispecific targeting agent to its target can be adjusted to obtain a specific effect. In addition to being able to bring two or targets together, ideally, new technologies can be useful for stimulating the targets in desired ways.

### SUMMARY OF INVENTION

The present inventors have found that the limitations described above can be overcome with a bispecific targeting agent comprising at least two targeting arms which can be produced separately to produce a properly-folded product that at high yield. These targeting arms can then be linked together by a nanoparticle or a liposome wherein:

a first targeting arm of the bispecific targeting agent is capable of specifically binding to a target antigen expressed on one cell,

a second targeting arm of the bispecific targeting agent is capable of specifically binding to a target antigen expressed on another cell,

ratios between targeting arms can be varied and can range from 1000 or more to 1 between distinct types of targeting arms.

Any cell or antigen that can be targeted can be subject to targeting by the disclosed targeting agents. For example, the cell types that may be recruited by the bispecific targeting agent may be both human or one of the cells may be human and the other an infectious agent. The platform is based on an empty nanoparticle or liposome conjugated to two or more targeting moieties, bound to the nanoparticle/liposome at defined ratios that may be other than 1:1. Such, compositions provide for specific binding to each cell type. This bispecific targeting agent can further be linked to growth factors or cytokines to further potentiate the effect of the bispecific targeting agent as a therapeutic or to exert a specific biologic effect on one or both cells being targeted.

According to one embodiment of this first aspect of the invention, each targeting arm is an antibody.

According to another embodiment of the first aspect of the invention, each targeting arm is a single polypeptide chain.

According to yet another embodiment of the first aspect of the invention, the first targeting arm is a single polypeptide chain and the second targeting arm is an antibody.

Additional targeting arms can also be included in additional embodiments.

In a second aspect a bispecific targeting agent comprising two targeting arms and a recombinant protein able to exert a specific activity on two cells being brought together by said targeting agent is disclosed wherein:

a first targeting arm of the bispecific targeting agent is capable of recruiting the activity of one human cell by specifically binding to an antigen expressed on one cell,

a second targeting arm of the bispecific targeting agent is capable of specifically binding to a target antigen expressed on another cell,

a recombinant protein with stimulatory activity including but not limited to growth factors or cytokine can also be linked to the liposome or a nanoparticle linker wherein the growth factor or cytokine is capable of stimulating or inhibiting the biological activity of one or both cells being targeted by the bispecific liposome.

In certain embodiments of the second aspect, a bispecific targeting agent is linked to a cytokine or growth factor containing at least two targeting arms which are both antibodies.

In certain embodiments of the second aspect, the bispecific targeting agent can be linked to a cytokine or growth factor wherein the two targeting arms are both single polypeptide chain antibodies.

According to another embodiment of the second aspect, the bispecific targeting agent can be linked to a cytokine or growth factor wherein the two targeting arms include a single polypeptide chain and an antibody.

The greater simplicity in molecular design of the disclosed targeting agents can provide for simplified production and can ensure proper folding of the targeting arms. For example, the bispecific nanoparticle/liposome targeting arms can be produced by conventional, well understood expression systems such as CHO or (E. coli for simpler single chain polypeptides) at high titers. These arms can then be linked together in desired ratios to a nanoparticle or liposome to provide a tailorable product attribute that was not previously possible with known bispecific products.

### BRIEF DESCRIPTION OF FIGURES

FIG. 1 provides an illustration of a bispecific targeting agent containing a cytokine consisting of one antibody binding to immune cell and the second antibody binding a cancer cell conjugated to a liposomal or polymeric linker.

FIG. 2 provides an illustration of a bispecific targeting agent consisting of one antibody binding to an immune cell and the second antibody binding a cancer cell, all conjugated to a liposomal or polymeric linker.

FIG. 3 provides an illustration of a bispecific targeting agent consisting of one antibody binding to an immune cell and the second antibody binding an infected cell, both conjugated to a liposomal or polymeric linker

FIG. 4 provides an illustration of a bispecific targeting agent containing a cytokine consisting of one antibody binding to an immune cell and the second antibody binding an infected cell, all conjugated to a liposomal or polymeric linker.

FIG. 5A provides a photograph of an SDS-PAGE (R-reduced and NR-non-reduced of anti-CD56 and anti-HER2 antibodies).

FIG. 5B provides a photograph of an experiment that shows binding of 5:1 and 1:5 anti-HER2:anti-CD56 bispecific variants to HER2 or CD56 proteins.

FIG. 6 provides data showing recruitment of NK cells to HER-2 positive cancer cells using FACS experiments with CFSE labeled SKBR-3 cells and APC labeled NK cells purified from PBMC cells.

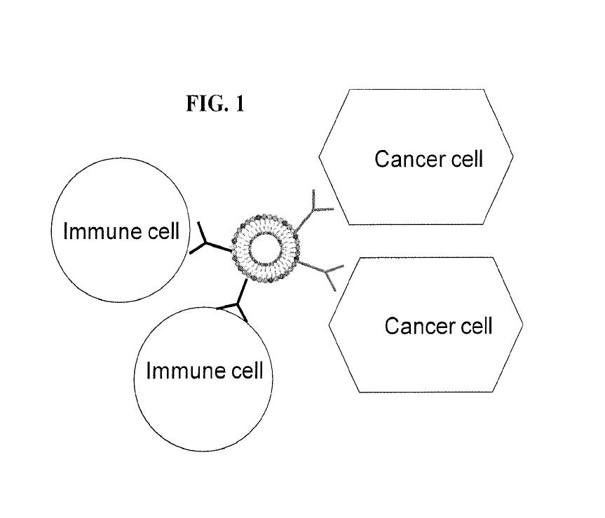

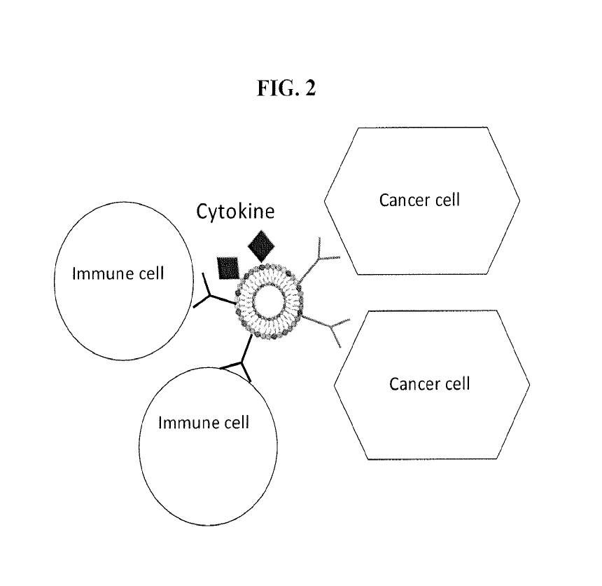

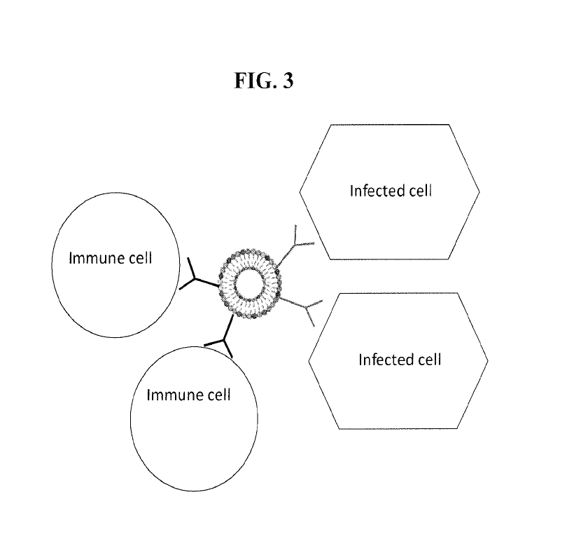

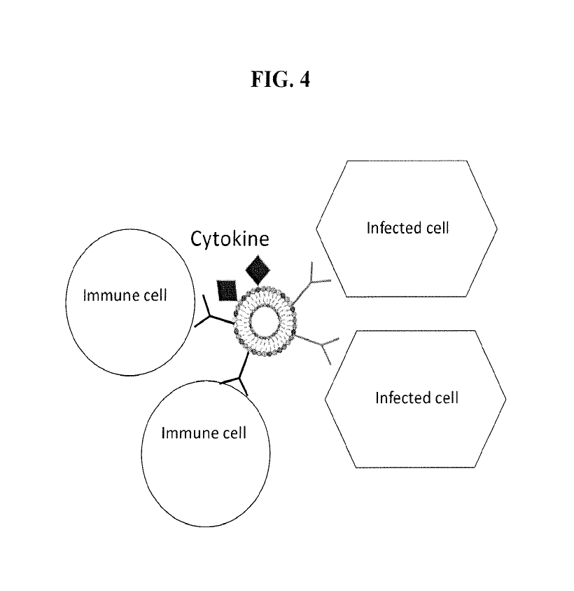

### DETAILED DESCRIPTION

As used herein the phrase “bispecific targeting agent” means a therapeutic product capable of binding two or more antigens. The targeting arms can be held together using a nanoparticle or liposome linker (bispecific linker) via covalent and noncovalent bonds or entirely by noncovalent bonds.

As used herein the phrase “targeting arm” means a component of the bispecific targeting agent that binds a targeted moiety. Generally, the targeting agents will be proteins including but not limited to single chain antibodies, antibody fragments or antibodies, including monoclonal antibodies.

For purposes of the present disclosure the molar ratio of any two targeting arms will depend on the efficacy and the toxicity of the resulting bispecific targeting agent. For example, a highly toxic targeting arm may be 1/100th of the amount of the other targeting arm. The exact ratio for any embodiment can be determined empirically. It is envisioned that typical ratios will be in the range of about 1:100 to 1:1, or 1:1 to 1:100 more typically the ratio will be in the range of about 1:10 to about 1:1, or about 5:1 to about 1:1 or about 3:2 to about 1:1 or about 1:1.

In certain embodiment a modulator can be included. As used herein a modulator is a component of the bispecific targeting agent that can affect properties of nearby cells including inducing or inhibiting cell growth, recruitment, inducing apoptosis. Exemplary modulators include cytokines, and growth factors of which many examples are known.

Methods are disclosed for using liposomes or nanoparticles to link various targeting moieties to produce bi- or multi-specific targeting agents.

In certain embodiments the liposome or nanoparticle linker also contains a cytokine or growth factor.

Methods for preparing bi- or multi-specific therapeutic agents by linking two or more targeting moieties to the liposome or nanoparticle are also disclosed.

Methods for preparing bi- or multi-specific targeting agents by linking two or more targeting moieties to a liposome at specific, predefined ratios which may not be 1 are also disclosed. This has not been found possible using previously known bispecific antibody preparation techniques.

In certain embodiments bi- or multi-specific targeting agents comprising two or more targeting arms are covalently linked to a liposome that can then bind a combination of cell types.

In certain embodiments bi- or multi-specific targeting agents comprising two or more targeting arms are linked to a liposome and the liposome also carries additional cytokines or growth factors that can stimulate cells that when bound by the targeting agent.

The use of such targeting agents for the preparation of pharmaceutical compositions is also contemplated.

Methods for the prevention, treatment or amelioration of diseases comprising administration of an effective amount of such the disclosed targeting agents are also disclosed.

Methods for the prevention, treatment or amelioration of cancer are also disclosed wherein one targeting arm of the bispecific targeting agent specifically binds to an antigen present on a cancer cell and another targeting arm specifically binds to an antigen on immune cell which include, but are not limited to, lymphocytes (T cells, B cells, NK cells), macrophages, and/or granulocytes.

Method for the prevention, treatment or amelioration of cancer are also disclosed wherein one targeting arm specifically binds to an antigen present on a cancer cell and another targeting arm specifically binds to antigen on immune cell which include but are not limited to, lymphocytes (T cells, B cells, NK cells), macrophages, and/or granulocytes.

Methods for the treatment or amelioration of infectious agent are disclosed wherein one targeting arm of the targeting agent specifically binds to an antigen present on an infectious agent or infected cell and another targeting arm specifically binds to an antigen on an immune cell which may include, but are not limited to, lymphocytes (T cells, B cells, NK cells), macrophages, and/or granulocytes.

Methods for the treatment or amelioration of infectious agents are disclosed wherein one targeting arm of the bispecific targeting agent specifically binds to an antigen present on an infectious agent or an infected cell and another targeting arm specifically binds to an antigen on immune cell including, but not limited to, lymphocytes (T cells, B cells, NK cells), macrophages, and/or granulocytes, following which the stimulating factor (e.g., cytokine) increases immune cell activity or cell proliferation to kill the infectious agent.

The disclosed methods have at least two highly advantageous effects. First, larger amounts of the each targeting arm can be produced in functional form per batch than previously possible for single chain bispecific antibodies allowing for greater efficiency and, ultimately, economy in production. Second, a greater number of constructs in the format of the bi- or multi-specific targeting agent can be constructed and considered as therapeutic candidates since any targeting moieties can be added once it is produced.

At the same time, the disclosed simpler construction methods avoid potential undesired intermolecular associations as well as reduce immunogenicity that can be present when prior art methods are used.

### EXAMPLES

### Example 1—Production of Bispecific Liposomal Targeting Agent

This example demonstrates one method for producing a bispecific liposomal targeting agent.

Liposome linker can consist of 40-60 weight percent phospholipid, 10-50 weight percent cholesterol, and 10-50 weight percent lipid-anchored poly(ethylene glycol) with a terminal azide or alkyne group. The lipid tails can range from 10 carbons to 18 carbons in length, with the PEG chain ranging from 350 Da to 10 kDa in size. The lipid solutions can be solubilized in ethanol and mixed to form the organic solution. The aqueous solution can consist of 1×PBS. The organic layer can be added to the rapidly stirring aqueous layer at a volume ratio of 1:2. After stirring the liposomal solution can be extruded to obtain particles between 40 nanometers and 3 microns in diameter.

The liposomal particles can be dialyzed against IX PBS to remove any ethanol. Post dialysis, the liposomes can be incubated at room temperature with equal amounts of two distinct targeting agents either antibodies or single chains, one targeting a tumor cell and the other targeting either CD8+ T cells or NK cells. The antibodies can be terminally labeled with either an azide or an alkyne group, and combined at a 1:1 molar ratio with the lipid-anchored PEG, covalently bonding via copper free click chemistry.

After conjugation any excess antibodies are removed, such as via size exclusion chromatography or other UF/DF method.

### Example 2—Production of Bispecific Polymeric Nanoparticle Targeting Agent

This example demonstrates a method for the production of a bispecific polymeric nanoparticle targeting agent.

The nanoparticle linker can consist of a hyperbranched poly(beta amino ester) (HBAE) and poly(ethylene glycol) (PEG) block co-polymer. The weight ratio for HBAE to PEG can range from about 1:10 up to about 10:1, with HBAE forming the core with the PEG chains extending from the HBAE termini. The distal end of the PEG chain can terminate in either an azide or an alkyl group. The HBAE block can range from 500 Da to 50 kDa in size, with the PEG chains ranging from 500 Da to 50 kDa in size.

The polymer can be solubilized in ethanol and constitute the organic solution. The aqueous solution can consist of 1×PBS. The organic layer can be added to the rapidly stirring aqueous layer at a volume ratio of 1:2. After stirring the liposomal solution can be extruded to obtain particles between 40 nanometers and 3 microns in diameter.

The polymeric particles can be dialyzed against IX PBS to remove any ethanol. Post dialysis the particles can be incubated at room temperature with equal amounts of two distinct antibodies, one targeting a tumor cell and the other targeting either CD8+ T cells or NK cells. The antibodies can be terminally labeled with either an azide or an alkyne group, and combined at a 1:1 molar ratio with the PEG chains, covalently bonding via copper free click chemistry.

After conjugation any excess antibodies are removed, such as via size exclusion chromatography or other UF/DF method.

### Example 3—Bispecific Targeting Agent Comprising Anti-CD56 and Anti-HLA-G Antibodies Conjugated to a Liposomal or Polymeric Linker

This example demonstrates a bispecific targeting agent comprising anti-CD56 And Anti-HLA-G antibodies conjugated to a liposomal or polymeric linker. This is a bispecific targeting agent that has two targeting agents attached to the surface of a liposomal or polymeric linker wherein one antibody targets a cancer cell and the other may bind to immune system cells.

In this example, the bispecific targeting agent is produced by conjugating anti-CD56 and anti-HLA-G antibodies to a liposomal or polymeric particle via a covalent linker as described in example 1 and 2 respectively.

NK cells are natural killer cells effective at eliminating tumors and virus-infected cells. In addition, NK (Natural Killer) cells are an important source of cytokines that activates CD8+ T cells, NK cells and macrophages. In many malignancies, NK cells have been shown to be inactivated, likely by receptors expressed specifically by the tumors, such as HLA-G. In other situations, the NK cells are inherently suppressed thereby allowing tumor escape and subsequent malignancy. The bi-specific targeting agent is aimed at activating tumor killing cells, such as NK cells, by blocking inhibitory receptors expressed by both tumors and NK cells.

CD56 is expressed by most NK cells. HLA-G is expressed by tumors to inhibit NK cell responses. The CD56 and HLA-G bispecific targeting agent recruits the NK cell to the tumor and reduces tumor inhibitory functions of both the NK cell and tumor which can release inflammatory cytokines to kills the cancer cells.

### Example 4—Bispecific Targeting Agent Comprising Anti-KIR and Anti-HLA-G Antibodies Conjugated to a Liposomal or Polymeric Linker

To further illustrate the invention, a bispecific targeting agent comprising anti-KIR and anti-HLA-G antibodies conjugated to a liposomal or polymeric linker is designed to bridge the KIR expressing NK cell and HLA-G expressing tumor cell. The antibodies are conjugated to a liposomal or polymeric particle via a covalent linker as described in example 1 and 2, respectively.

NK cells are natural killer cells effective at eliminating tumors and virus-infected cells. In addition, NK (Natural Killer) cells are important source of cytokines that activate CD8+ T cells, NK cells and macrophages. In many malignancies, NK cells have been shown to be inactivated, likely by receptors expressed specifically by the tumors, such as HLA-G. In other situations, the NK cells are inherently suppressed thereby allowing tumor escape and subsequent malignancy. The bi-specific targeting agent is aimed at activating tumor killing cells, such as NK cells, by blocking inhibitory receptors expressed by both tumors and NK cells.

### Example 5—Bispecific Targeting Agent Comprising Anti-NKG2 and Anti-HLA-G Antibodies Conjugated to a Liposomal or Polymeric Linker

Example 5 provides a bispecific targeting agent comprising anti-NKG2 and anti-HLA-G antibodies conjugated to a liposomal or polymeric linker. The anti-NKG2 and anti-HLA-G antibodies are conjugated to a liposomal or polymeric particle linker via a covalent linkage as described in Examples 1 and 2, respectively. This targeting agent is designed to bridge the NKG2 expressing NK cell and HLA-G expressing tumor cell.

NK cells are natural killer cells effective at eliminating tumors and virus-infected cells. In addition, NK (Natural Killer) cells are an important source of cytokines that activate CD8+ T cells, NK cells and macrophages. In many malignancies, NK cells have been shown to be inactivated, likely by receptors expressed specifically by the tumors, such as HLA-G. In other situations, the NK cells are inherently suppressed thereby allowing tumor escape and subsequent malignancy. This bispecific targeting agent will activate tumor killing cells, such as NK cells, by blocking inhibitory receptors expressed by both tumors and NK cells.

### Example 6—Bispecific Targeting Agent Comprising Anti-NKG2 and Another Targeting Agent to a Cancer Cell Conjugated to a Liposomal or Polymeric Linker

This example demonstrates a bispecific targeting agent comprising anti-NKG2 and another targeting agent to a cancer cell conjugated to a liposomal or polymeric linker In this example, a bispecific targeting agent can target NKG2, an inhibitory receptor expressed by NK cells, and one of the tumor cells bearing tumor antigens, including but not limited, to MAGE-1 (melanoma), MUC-1 (colon, breast, ovarian, lung and pancreatic cancers), EPCAM, or Her-2 (breast cancer) to bridge the NK cell and tumor cell and also allow activation of NK cells and subsequent killing of tumor cells. The antibodies are conjugated to a liposomal or polymeric particle via a covalent linker as described in example 1 and 2, respectively.

NK cells are natural killer cells effective at eliminating tumors and virus-infected cells. In addition, NK (Natural Killer) cells are important source of cytokines that activate CD8+ T cells, NK cells and macrophages. In many malignancies, NK cells have been shown to be inactivated, likely by receptors expressed specifically by the tumors, such as HLA-G. In other situations, the NK cells are inherently suppressed thereby allowing tumor escape and subsequent malignancy. The bi-specific targeting agent is aimed at activating tumor killing cells, such as NK cells, by blocking inhibitory receptors expressed by both tumors and NK cells.

### Example 7—Bispecific Targeting Agent Comprising Anti-KIR and One Targeting Agent to a Cancer Cell Conjugated to a Liposomal or Polymeric Linker

This example demonstrates a bispecific targeting agent that can target KIR inhibitory receptor and tumor cell antigens, including but not limited, to MAGE-1 (melanoma), MUC-1 (colon, breast, ovarian, lung and pancreatic cancers), EpCAM or Her-2 (breast cancer) or both. The antibodies are conjugated to a liposomal or polymeric particle via a covalent linker as described in example 1 and 2, respectively.

NK cells are natural killer cells effective at eliminating tumors and virus-infected cells. In addition, NK (Natural Killer) cells are important source of cytokines that activate CD8+ T cells, NK cells and macrophages. In many malignancies, NK cells have been shown to be inactivated, likely by receptors expressed specifically by the tumors, such as HLA-G. In other situations, the NK cells are inherently suppressed thereby allowing tumor escape and subsequent malignancy. The bi-specific targeting agent is aimed at activating tumor killing cells, such as NK cells, by blocking inhibitory receptors expressed by both tumors and NK cells.

### Example 8—Bispecific Targeting Agent Comprising Anti-CD8 and Anti-HER2 to a Cancer Cell Conjugated to a Liposomal or Polymeric Linker

In this specific example bispecific targeting construct can bridge CD8+ T cells by anti-CD8 antibody with HER2 positive tumor breast, gastric or ovarian cell (or other).

CD8+ T cells (CTLs) detect tumor cells or virally infected cells through the major histocompatibility complex (MHC), which presents viral or tumor antigens. CTLs are therefore antigen-specific and recognize peptides derived from virus or tumor antigens. CTLs kill their target cells by cytotoxic molecules which are stored in secretory lysosome as well as inducing apoptosis.

### Example 9—Bispecific Targeting Agent Comprising Anti-CD8 and One Targeting Agent to a Cancer Cell Conjugated to a Liposomal or Polymeric Linker

This example demonstrates a bispecific targeting construct that can bridge CD8+ T cells by anti-CD8 antibody to the tumor antigen on the tumor cells including but not limited, to MAGE-1 (melanoma), MUC-1 (colon, breast, ovarian, lung and pancreatic cancers), or EpCAM. The antibodies are conjugated to a liposomal or polymeric particle via a covalent linker as described in example 1 and 2 respectively.

CD8+ T cells (CTLs) detect tumor cells or virally infected cells through the major histocompatibility complex (MHC), which presents viral or tumor antigens. CTLs are therefore antigen-specific and recognize peptides derived from virus or tumor antigens. CTLs kill their target cells by cytotoxic molecules which are stored in secretory lysosome as well as inducing apoptosis.

### Example 10—Bispecific Targeting Agent Comprising Anti-CD8 and HER-2 to a Cancer Cell Conjugated to a Liposomal or Polymeric Linker Carrying a IL-15 Cytokine

This example demonstrates an immune cell stimulating cytokine such as IL-15 coupled to the surface of the bi-specific targeting agent. IL-15 drives both NK cell activation and CD8+ cytolytic T cell activation without simultaneous regulatory T cell expansion. The antibodies are conjugated to a liposomal or polymeric particle via a covalent linker as described in example 1 and 2 respectively.

CD8+ T cells (CTLs) detect tumor cells or virally infected cells through the major histocompatibility complex (MHC), which presents viral or tumor antigens. CTLs are therefore antigen-specific and recognize peptides derived from virus or tumor antigens. CTLs kill their target cells by cytotoxic molecules which are stored in secretory lysosome as well as inducing apoptosis.

### Example 11—Bispecific Targeting Agent Preparation Method

The following example demonstrates one method of preparing the bispecific targeting agent by first preparing a bispecific linker and then attaching the proteins directly through active cysteine or though free thiols that have been introduced to primary amines on the recombinant protein by a thiolation reaction.

Step 1. Preparation of a Bispecific Liposome Linker:

Prepare 1,2-distearoyl-sn-glycero-3-phosphoethanolamine-N-[maleimide(polyethylene glycol)-2000] DSPE-PEG₂₀₀₀-Maleimide solution by weighing a small amount of DSPE-PEG₂₀₀₀-Maleimide and dissolve it in absolute ethanol. Solvents other than ethanol can also be used so long as the lipid is sufficiently soluble as is known. Other activated lipids can also be used instead of DSPE-PEG₂₀₀₀-Maleimide. Other reactive lipids can also be used. These types of lipids are known to one skilled in the art of nanoparticle conjugation.

Combine the following lipid solutions of hydrogenated soy phosphatidylcholine (HSPC), cholesterol, DSPE-PEG₂₀₀₀ and DSPE-PEG₂₀₀₀-Maleimide. Other combinations of lipids can also be used followed by the addition of absolute ethanol to lipid solution.

Prepare an aqueous solution such as PBS in a separate container.

Under controlled speed, add lipid solution to aqueous solution while mixing to form liposomes of a desired size.

Extrude until the desired particle size is reached at appropriate temperature 55-70° C. using stacked 200/200/100/100 nm membranes, with drain discs in between following by stacked 100/100/80/80 nm membranes with drain discs in between and if desired with stacked 80/80/50/50 nm membranes with drain discs in between. Other membrane combinations can also be used.

Determine the size of the liposome using the Malvern zetasizer. Generally bispecific linkers (nanoparticle or liposome) can vary in size based on the application.

Buffer can be exchanged to any desired concentration and buffer so long as the buffer does not interfere with the thiolation step using. Tangential flow filtration or equivalent or other methods can be used.

Step 2. Attachment of Targeting Arms to the Bispecific Linker:

Depending on the recombinant protein being conjugated to the bispecific linker, different methods can be used. The most straight forward method is to engineer the recombinant proteins such that a single free cysteine is available for attachment to DSPE-PEG₂₀₀₀-Maleimide present on the bispecific linker. If this is not possible, in the second method recombinant proteins including but not limited to antibodies can be attached to the bispecific linker by thiolation. Recombinant protein thiolation is accomplished using Traut's reagent (2-iminothiolane) which attaches a sulfhydryl group to primary amines, while still keeping charge properties similar to the original amino group. After proper reaction of Traut's reagent with the recombinant protein, the thiolated recombinant protein is then added to the bispecific linker containing an activated lipid such as PEG-DSPE-maleimide capable of conjugating to the thiolated antibody. After allowing the reaction to take place, free thiols on the protein are removed by capping reaction with L-cysteine. Following the capping reaction, unreacted antibodies are removed from the bispecific product by tangential flow filtration or other equivalent methods using a membrane greater than the size of the recombinant proteins being conjugated. In case of antibodies a membrane 300,000 MW can be used.

Step 3. Targeting Arm Thiolation and Conjugation:

Dissolve Traut's reagent in 0.1M sodium bicarbonate pH 8.5 (solvent A) or equivalent.

Prepare two or more protein solutions at concentrations between 0.5-20 mg/ml to make the composition bispecific or multispecific.

Empirically establish the molar excess of Traut's reagent that produces best conjugation efficiency for each protein. Each protein may require a different ratio. 10-fold molar excess of Traut's reagent to the antibody solution can be used as a starting ratio.

Preferably an EDTA solution is used to chelate divalent metals in solution and prevent oxidation of sulfhydryl groups.

Allow the reaction to proceed for example by incubation at room temperature for 1 hr. Stop the reaction by buffer exchange or my addition of excess L-Lysine.

Buffer exchange to 25 mM HEPES, or 1×PBS, 150 mM NaCl, pH 7.0 for 4 hrs at ambient temperature (or overnight at 2-8C) using Tangential Flow Filtration (TFF) or equivalent method

Determine protein concentration of the protein samples.

Determine the ratio of each protein to be conjugated. For example a bispecific targeting agent can be made by mixing two different antibodies at 1:1 ratio which is typical for other bispecific targeting agent or at custom ratio, for example 5:1, 1:5, 3:2 and others to change the efficacy of the interaction of the bispecific targeting agent with a given cell.

Determine the number of moles of the bispecific linker using the equation below, where r is the radius of the nanoparticle in nanometers (this can be calculated on an excel formulation worksheet).

$$moles of liposomes = \frac{\frac{total number of phospholipids}{\frac{4 π ( {r}^{2} - {( r - 5 )}^{2} )}{0.71}}}{6.02 \times {10}^{23}}$$

Empirically establish the ratio of the recombinant proteins to the bispecific linker. A good starting ratio is 50:1 mole ratio of antibody to the bispecific linker prepared with DSPE-PEG-Maleimide.

Incubate the mixture of linker and proteins to allow the reaction to proceed to completion.

Terminate the incubation by adding a volume of 500 mM L-Cysteine. The optimal concentration to be determined empirically. A concentration of 15 mM is a good starting point.

Remove the unconjugated proteins using tangential flow filtration or other equivalent methods using a membrane greater than the size of the recombinant proteins being conjugated. In case of antibodies a membrane 300,000 MW can be used.

### Example 12 Preparation of Anti-HER2 and Anti-CD56 Bispecific Targeting Agent

This example demonstrates the preparation of anti-HER2:anti-CD56 Bispecific Product using methods described in Example 11.

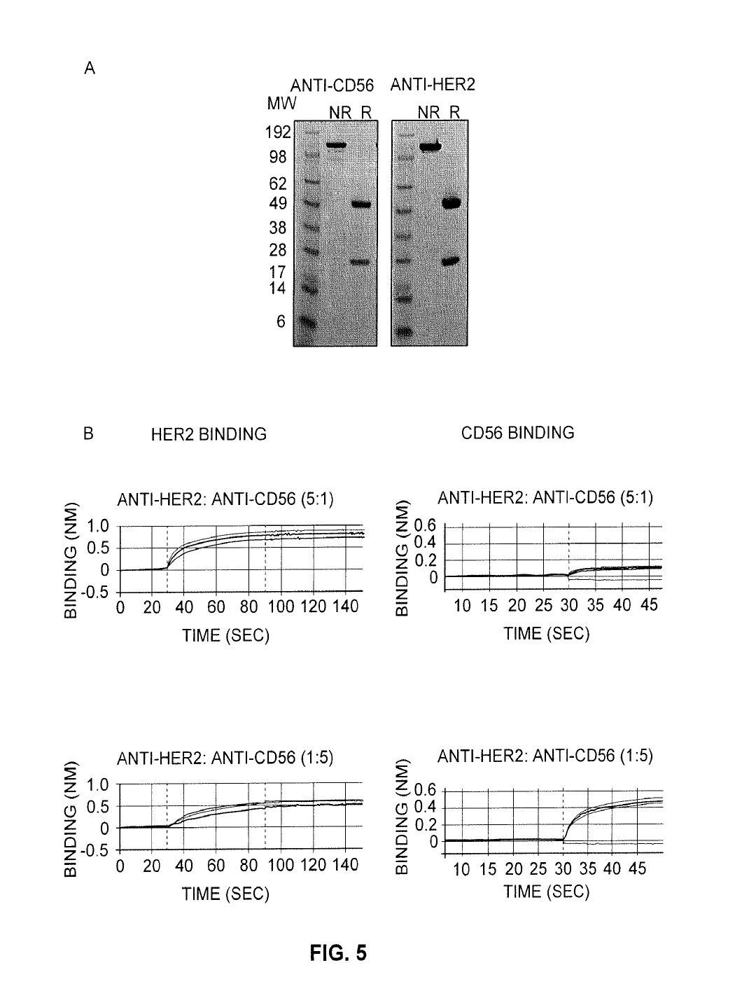

Three different bispecific targeting variants containing anti-HER2 and anti-CD56 were prepared by varying the ratio of two targeting arms (antibodies:anti-HER2 or anti-CD56) prior to their conjugation to the linker as described in Example 11. The ratios of each targeting arm (antibody) in this experiment were 1:1, 5:1 and 1:5 for Anti-HER2 and anti-CD56, respectively, however other variants may be possible and more efficacious. The purity of the antibodies is shown in FIG. 5A.

Binding of each bispecific targeting variant to CD-56 or HER2 was determined using Fortebio instrument (FIG. 5B shows data for variants 5:1, 1:5 but not for 1:1). FIG. 5B shows that both bispecific targeting product variants are able to bind HER2 and CD56 proteins. Bispecific targeting variant anti-HER2:anti-CD56 (5:1) showed slightly higher binding to HER2 and lower affinity for CD-56. Conversely variant anti-HER2:anti-CD56 (1:5) showed higher binding to CD56 and slightly lower to HER2. While CD-56 binding was affected by different concentrations of anti-CD56 antibody conjugated to the linker, anti-HER2 antibody showed similar binding at both concentrations. This is most likely because anti-HER2 antibody has higher affinity to its target as compared to the anti-CD56 antibody. In summary, data shown in FIG. 5 shows that the method described in Example 11 allows one to produce a bispecific product with different binding potential to the different targets by varying the amount of antibody being conjugated to the bispecific linker.

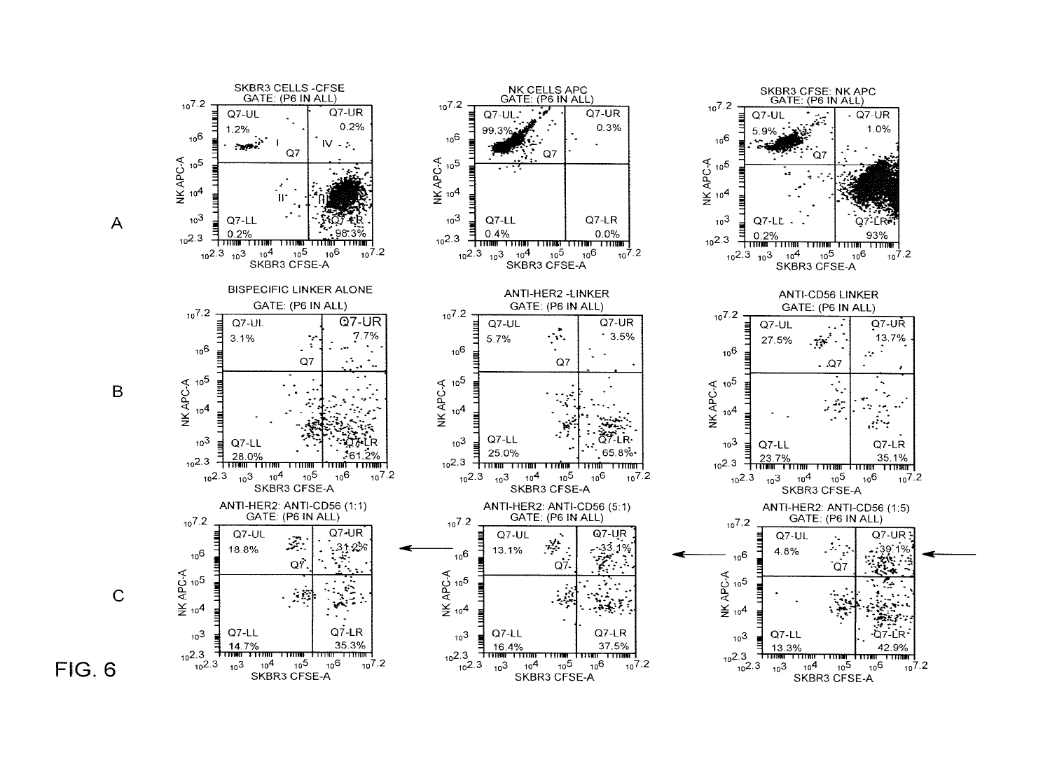

Additional experiments have established the ability of the bispecific targeting agent to recruit CD-56 positive Natural Killer cells (NK) purified by negative magnetic selection to the HER-2 positive SKBR3 breast cancer cells. To visualize the ability of the bispecific targeting agent to bridge both cell types SKBR3 cells were labeled with CFSE fluorescent dye which shifts the cells to quadrant III (FIG. 6A) while the NK cells were labeled with eFluor® 670 which shifts cells to quadrant I (FIG. 6A, middle figure). When both cells are mixed together (FIG. 6A) right panel they are visibly separated in two different quadrants. In these experiments we are looking for a shift of NK cells from quadrant I to IV and for SKBR3 cells from Quadrant III to IV. In this experiment mixed cell population were incubated at 3:1 (NK cells to SKBR3 ratio) for 3 hours at 37 C in RPMI 1640 in the presence of different bispecific targeting agents and controls. Cells were fixed with 0.1% paraformaldehyde and data was acquired on Accuri flow cytometer. This experiment demonstrated that all bispecific targeting agents increased the recruitment of the NK cells to the SKBR3 cells (31-39%) as compared to the controls (3.5-13.7%).

1-38. (canceled)

39. A multispecific targeting agent comprising at least two targeting arms linked together by a liposome wherein:
a first targeting arm of the targeting agent binds a first cell by binding to an antigen expressed on the cell,
a second targeting arm of the targeting agent binds a target antigen expressed on a second cell, and
further comprising a recombinant protein with stimulatory or inhibitory activity that is capable of stimulating or inhibiting the biological activity of the cells being targeted by the bispecific targeting agent.

40. The targeting agent of claim 39 wherein the recombinant protein is a growth factor.

41. The targeting agent of claim 39 wherein the recombinant protein is a cytokine.

42. The targeting agent of claim 39 wherein the recombinant protein is IL-15.

43. The targeting agent of claim 39 wherein the recombinant protein is IL-2.

44. The targeting agent of claim 39 wherein the targeting arms are present in a ratio other than 1:1.

45. The targeting agent of claim 39 wherein both targeting arms comprise the same antibody.

46. The targeting agent of claim 39 wherein each targeting arm is an antibody.

47. The targeting agent of claim 39 wherein each targeting arm is a single chain antibody.

48. The targeting agent of claim 39 wherein one targeting arm is a single chain antibody or antibody that binds CD-56.

49. The targeting agent of claim 39 wherein one targeting arm is a single chain antibody or antibody that binds NKG2.

50. The targeting agent of claim 39 wherein one targeting arm is a single chain antibody or antibody that binds KIR.

51. The targeting agent of claim 39 wherein one targeting arm is a single chain antibody or antibody that binds CD-8.

52. The targeting agent of claim 39 wherein one targeting arm is a single chain antibody or antibody that binds HLA-G.

53. The targeting agent of claim 39 wherein one targeting arm is a single chain antibody or antibody that binds HER-2.

54. The targeting agent of claim 39 wherein one targeting arm is a single chain antibody or antibody that binds MUC1.

55. The targeting agent of claim 39 wherein one targeting arm is a single chain antibody or antibody that binds EpCAM.

56. The targeting agent of claim 39 wherein one targeting arm is a single chain antibody or antibody that binds MAGE-1.

57. The targeting agent of claim 39, wherein the liposome linker comprises a phospholipid, cholesterol, and lipid-anchored polyethylene glycol having a terminal azide or alkyne group.

58. The targeting agent of claim 39, wherein the liposome linker comprises 40-60 weight percent of a phospholipid, 10-50 weight percent of a cholesterol, and 10-50 weight percent of a lipid-anchored polyethylene glycol having a terminal azide or alkyne group.

59. The targeting agent of claim 39, wherein the lipid includes a tail that ranges from 10 carbons to 18 carbons in length, with the polyethylene glycol chain ranging from 350 Da to 50,000 Da in size.

60. The targeting agent of claim 39, wherein the liposome comprises a particle between 40 to 500 nanometers.

61. The targeting agent of claim 39, wherein the antibodies are terminally labeled with an azide, NHS, alkyne or a maleimide group.

62. A method for the prevention, treatment or amelioration of a disease or disease-causing cell comprising administration of an effective amount of the targeting agent of claim 39.

63. The method for the prevention, treatment or amelioration of a disease of claim 62 wherein the disease or disease-causing cell is a cancer cell.

64. The method for the prevention, treatment or amelioration of a disease of claim 62 wherein the disease or disease-causing cell is an infectious agent that causes an infection or an infected cell.

65. The method for the prevention, treatment or amelioration of a disease or disease-causing cell of claim 62 wherein one targeting arm of the targeting agent specifically binds to an antigen on an immune cell comprising lymphocytes (T cells, B cells, NK cells), macrophages, or granulocytes.
